(19) United States
(12) Patent Application Publication
(10) Pub. No.: US 2010/0279308 A1
(43) Pub. Date: Nov. 4, 2010

(54) RAPID LATERAL FLOW GLYCAN-DETECTING DEVICE

(76) Inventors: Ardythe L. Morrow, Cincinnati, OH (US); David S. Newburg, Newtonville, MA (US); Guillermo Ruiz-Palacios, Mexico City (MX)

Correspondence Address: OCCHIUTI ROHLICEK & TSAO, LLP, 10 FAWCETT STREET, CAMBRIDGE, MA, 02138, US

(21) Appl. No.: 12/719,511

(22) Filed: Mar. 8, 2010

Related U.S. Application Data
(60) Provisional application No. 61/157,933, filed on Mar. 6, 2009.

(51) Int. Cl. G01N 33/53; C12M 1/34

(52) U.S. Cl. 435/7.1; 435/287.2

(57) ABSTRACT

A glycan-detecting device containing a sample pad, a membrane in communication with the sample pad, a labeled lectin, an immobilized lectin of the same type, and an immobilized antibody specific to the lectin.

### RELATED APPLICATION

This application claims priority to U.S. Provisional Application No. 61/157,933, filed on Mar. 6, 2009, the content of which is hereby incorporated by reference in its entirety.

### BACKGROUND OF THE INVENTION

Galactoside 2-alpha-L-fucosyltransferase 2 (FUT2), encoded by the secretor gene FUT2, is responsible for synthesis of α1,2-fucosylated (secretor) glycans. Approximately 20-25% of individuals in Africa, America, Asia, and Europe lack an active FUT2 gene and are therefore incapable of expressing secretor glycans.

### SUMMARY OF THE INVENTION

One aspect of the present invention relates to a rapid lateral flow device for detecting a glycan. This device contains a sample pad, a membrane (e.g., a nitrocellulose membrane) in communication with the sample pad, a lectin-label conjugate, an immobilized lectin, and an immobilized anti-lectin antibody. The lectin in the conjugate is identical to the immobilized lectin. Also, the immobilized antibody specifically binds to this lectin. Suitable lectins for this device include, but are not limited to, UEA1, AIA, GSA II, WGA, sWGA, SNA, MAL-II, PWA, SJA, LEA, and I-PHA. The immobilized lectin and the immobilized anti-lectin antibody are located on the membrane at a first zone and a second zone, which do not overlap. The conjugate is located between the sample pad and either the immobilized lectin or the immobilized anti-lectin antibody. In one example, these four components are organized in the order of the sample pad, the conjugate, the immobilized lectin, and the immobilized anti-lectin antibody.

The device of this invention can further contain a support member, on which the sample pad and the membrane are mounted. This device can also contain a sink pad, which is separated from the sample pad by the membrane. In one example, the sample pad, the membrane, and the sink pad are sequentially mounted on the support member.

Also within the scope of this invention is a method of identifying a glycan expression phenotype in a subject using the device described above. The method includes (i) dispensing a bodily fluid (e.g, saliva) from a subject into the sample pad in the device, (ii) examining a signal at the first and second zones in the device, and (iii) determining a glycan expression phenotype in the subject based on the presence or absence of the signal at the first and second zones. When the signal is detectable at both zones, it indicates that the subject expresses a glycan capable of binding to the lectin in the device. If the signal is detectable only at the second zone, it indicates that the subject does not express that glycan. When a device containing lectin UEA1 is used, (i) detection of a signal at both the first and second zones indicates that the subject is secretor glycan positive; and (ii) detection of a signal at only the second zone indicates that the subject is secretor glycan negative.

The details of one or more embodiments of this invention are set forth in the description below. Other features or advantages of the present invention will be apparent from the following drawings and an actual example, and also from the appended claims.

### BRIEF DESCRIPTION OF THE DRAWINGS

The drawings are first described.

FIG. 1 is a diagram of a rapid lateral flow device for determining secretor status.

FIG. 2 is a digital image of an immunochromatographic strip test device for testing secretor status in saliva.

### DETAILED DESCRIPTION OF THE INVENTION

Disclosed herein is a glycan-detecting device useful in a rapid diagnostic test to determine a subject's glycan expression phenotype, particularly, secretor glycan status.

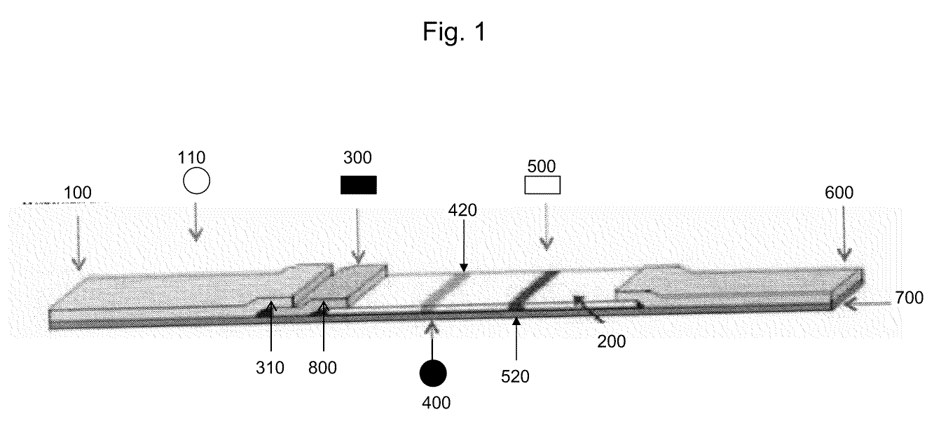

As shown in FIG. 1, the device of this invention includes sample pad 100, membrane 200, and, optionally, sink pad 600, all of which can be mounted sequentially on support member 700. Sample pad 100 is in communication with membrane 200. Namely, fluid placed on the sample pad is capable of traveling to the membrane. If desired, the device can further contain wick pad 310, located on top of membrane 200 for absorbing overflow fluid from sample pad 100.

Sample pad 100, sink pad 600, and wick pad 310 all can be made of a material capable of absorbing fluid, such as filter paper (e.g., Whatman GF/DVA membrane) or sponge.

Membrane 200 allows movement of biomolecules, e.g., proteins, nucleic acids, and polysaccharides. Materials suitable for making membrane 200 include, but are not limited to, nitrocellulose, nylon, cellulose, polyvinylidine fluoride (PVDF), polycarbonate, polypropylene, polyethylene, Teflon, and Kevlar. Support member 700, in either sheet or slab form, can be made of (i.e., containing) metal or plastic (e.g., styrene, polycarbonate, polypropylene, polyethylene, polyvinyl chloride).

The device of this invention further contains lectin 400, anti-lectin antibody 500, and lectin-label conjugate 300. Lectin 400 and anti-lectin antibody 500, both immobilized, are located at two separate zones 420 and 520 (i.e., zone 1 and zone 2) on membrane 200. Lectin-label conjugate 300, located between sample pad 100 and either lectin 400 or anti-lectin antibody 500, contains the same type of lectin as lectin 400 and a label, which can be any detectable marker. Examples of the label include, but are not limited to, colloidal gold, fluorescein and derivatives thereof (e.g. FITC—fluorescein isothyocyanate), rhodamine and derivatives thereof, green fluorescent protein (GFP) and derivatives thereof, quantum dots, and other fluorescent or chromophore molecules (e.g. dinitrophenyl hydrazine).

Lectins are sugar-binding proteins with high specificity to particular sugar moieties. Table 1 below lists exemplary lectins suitable for use in the device described herein and the sugar moieties to which they bind:

TABLE 1
Exemplary Lectins and Sugar Moieties to Which They Bind

| Source | Lectin | Sugar Moieties |
|---|---|---|
| Arthocarpus | AIA | α-D-Gal > Gal β(1-3)GalNAc terminal |
| integrifoli | | |
| Griffonia | GSA II | (1) res.term. α or β-GlcNAc |
| simplicifoliaII* | | |
| Triticum | WGA | Trimers GlcNAc β(1-4) > dimers > |
| vulgare* | | Neu5Ac- |
| Triticum | sWGA | Trimers GlcNAc β(1-4) > dimers |
| vulgare(suc)* | | GlcNAc. |
| Sambucus nigr | SNA | Neu5Ac-α(2-6)Gal y |
| | | Neu5Ac-α(2-6)GalNAc |
| Maackia | MAL-II | Neu5Ac-α(2-3)Gal |
| amurensi | | |
| Ulex europaeusI | UEA-I | Fuc α (1-2)Gal β (1.-4)GlcNAc > Fuc- |
| Phytolacca | PWA (PAA) | Oligomers of β(1-4)GlcNAc |
| American | | |
| Sophora japonica | SJA | GalNAc > Gal |
| Licopersicon | LEA | Oligomers of 4 [GlcNAc β(1-4)] |
| esculentu | | (no consecut.) |
| Phaseolus | 1-PHA | Galb(1-4)GlcNAc α(1-2)man |
| vulgaris leucoagl | | |

Immobilized antibody 500 is capable of binding to the lectin in the device. The term “antibody” is meant to include intact antibodies, antibody binding fragments, e.g., Fab and F(ab′)₂, and genetically modified antibodies, e.g., scFv antibodies, diabodies, and dual variable domain (DVD) Igs.

The anti-lectin antibody used in this invention can be prepared by conventional methods. See, for example, Harlow and Lane, (1988) Antibodies: A Laboratory Manual, Cold Spring Harbor Laboratory, New York. In general, a lectin can be isolated from its natural source or produced via recombination technology. To produce anti-lectin antibodies, the lectin, optionally coupled to a carrier protein (e.g., KLH), can be mixed with an adjuvant, and injected into a host animal. Antibodies produced in the animal can then be purified by affinity chromatography. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, CpG, surface-active substances such as lysolecithin, pluronic polyols, polyanions, peptides, oil emulsions, keyhole limpet hemocyanin, and dinitrophenol. Useful human adjuvants include BCG (bacille Calmette-Guerin) and Corynebacterium parvum. Polyclonal antibodies, i.e., heterogeneous populations of antibody molecules, are present in the sera of the immunized animal.

Monoclonal antibodies, i.e., homogeneous populations of antibody molecules, can be prepared using standard hybridoma technology (see, for example, Kohler et al. (1975) Nature 256, 495; Kohler et al. (1976) Eur. J. Immunol. 6, 511; Kohler et al. (1976) Eur J Immunol 6, 292; and Hammerling et al. (1981) Monoclonal Antibodies and T Cell Hybridomas, Elsevier, N.Y.). In particular, monoclonal antibodies can be obtained by any technique that provides for the production of antibody molecules by continuous cell lines in culture such as described in Kohler et al. (1975) Nature 256, 495 and U.S. Pat. No. 4,376,110; the human B-cell hybridoma technique (Kosbor et al. (1983) Immunol Today 4, 72; Cole et al. (1983) Proc. Natl. Acad. Sci. USA 80, 2026, and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD, and any subclass thereof. The hybridoma producing the monoclonal antibodies of the invention may be cultivated in vitro or in vivo. The ability to produce high titers of monoclonal antibodies in vivo makes it a particularly useful method of production.

In addition, techniques developed for the production of “chimeric antibodies” can be used. See, e.g., Morrison et al. (1984) Proc. Natl. Acad. Sci. USA 81, 6851; Neuberger et al. (1984) Nature 312, 604; and Takeda et al. (1984) Nature 314:452. A chimeric antibody is a molecule in which different portions are derived from different animal species, such as those having a variable region derived from a murine monoclonal antibody and a human immunoglobulin constant region. Alternatively, techniques described for the production of single chain antibodies (U.S. Pat. Nos. 4,946,778 and 4,704,692) can be adapted to produce a phage or yeast library of scFv antibodies. scFv antibodies are formed by linking the heavy and light chain fragments of the Fv region via an amino acid bridge.

Moreover, antibody fragments can be generated by known techniques. For example, such fragments include, but are not limited to, F(ab′)₂ fragments that can be produced by pepsin digestion of an antibody molecule, and Fab fragments that can be generated by reducing the disulfide bridges of F(ab′)₂ fragments.

Lectin 400 and anti-lectin antibody 500 can be immobilized on membrane 200 at non-overlapping zone 1 and zone 2 by a conventional method. Alternatively, they can be mounted on support member 700 at positions corresponding to zone 1 and zone 2. As mentioned above, lectin-label 300 is located between sample pad 100 and either lectin 400 or anti-lectin antibody 500. It can be placed on membrane 200 at a zone adjacent to sample pad 100. Alternatively, it is absorbed in pad 800 (see FIG. 1), which is in communication with both sample pad 100 and membrane 200. In one example, the device of this invention contains, sequentially, sample pad 100, lectin-label conjugate 300, lectin 400, anti-lectin antibody 500, and sink pad 600.

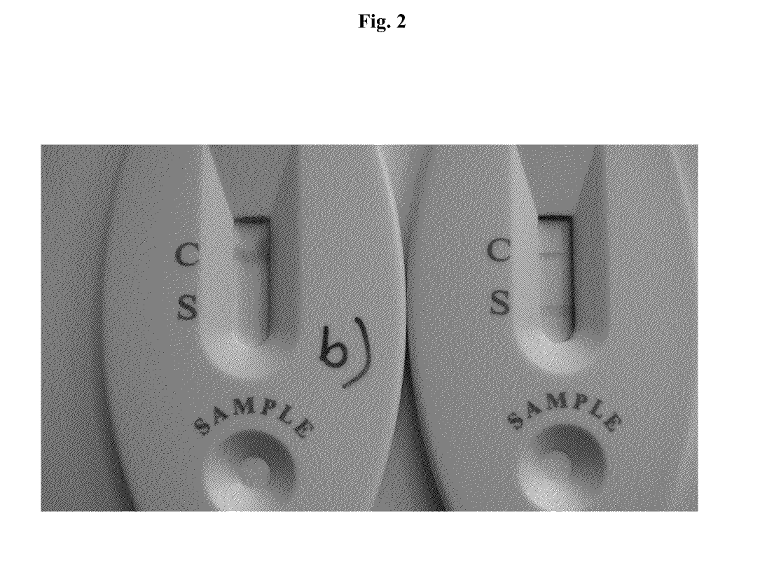

The device described above can be placed inside a cassette, with a portion of sample pad 100 and a portion of membrane 200 exposed. See FIG. 2. The exposed portion of sample pad 100 forms a sample well and the exposed portion of membrane 200 encompasses zone 1 and zone 2

The device of this invention is useful in detecting presence/absence of a particular type of glycan in a bodily fluid (e.g., saliva, milk, tears, blood, urine, seminal fluid, vaginal fluid, cerebrospinal fluid, synovial fluid, sweat, colostrum, or respiratory tract fluid). The type of glycan to be detected depends on the type of lectin contained in the device. For example, a device containing UEA1 is useful in examining secretor status of a subject, i.e., whether or not a subject (e.g., a human infant) expresses a secretor glycan.

To detect a glycan in a bodily fluid, sample 110 (see FIG. 1) from the fluid is dispensed into sample pad 100, in which the fluid spreads by way of wicking action. When the sample comes into contact with membrane 200, it mixes with lectin-lable conjugate 300 located between sample pad 100 and membrane 200. If the sample contains a glycan that binds to the lectin in conjugate 300, a glycan-conjugate complex would form. While moving along membrane 200, the sample contacts sequentially with immobilized lectin 400 and immobilized anti-lectin antibody 500 at zone 1 and zone 2, respectively. If the sample contains the glycan-conjugate complex mentioned above, this complex would bind to lectin 400, thereby generating a detectable signal at zone 1. Free lectin-label conjugate 300 binds to antibody 500, producing a detectable signal at zone 2. Thus, based on the signal at zone 1 or zone 2, presence/absence of the glycan in the bodily fluid can be determined. More specifically, if the signal is detectable at both zone 1 and zone 2, it indicates that the bodily fluid contains the glycan; and if the signal is detectable at zone 2, it indicates that the glycan is not present in the bodily fluid.

Applying the device of this invention, a subject's glycan expression phenotype can be determined within 15 minutes. Determining the glycan expression phenotype in an individual is useful to assess susceptibility to or risk for developing an infection to pathogens such as E. coli, Salmonella sp., Vibrio cholera, Neisseria gonorrhoeae, Clamydia trachomatis, Streptocococcus pyogenes, Streptococcus pneumoniae, Haemophilus influenza, Mycobacterium tuberculosis, Candida albicans, Coccidioides immitis, and others. See, e.g., Blackwell, C., 1989, FEMS Microbiology Immunology 47:341-350. Thus, the device described herein and its application are especially useful in rapid identification of individuals who are susceptible to or at risk for developing inflammatory and infectious disorders with serious morbidity and mortality, e.g., necrotizing enterocolitis (NEC), sepsis, and chorioamnionitis, in infants.

Described below is an example of using the rapid lateral flow device described herein for determining secretor status of infants.

In a lateral flow glycan-detecting device (see FIG. 1), a Pierce 0.45 μm nitrocellulose strip (200) were sensitized with UEA1 (400) at zone 1 (420) at a concentration of 2.5 μg/μL and with anti-UEA1 rabbit antiserum (500) at zone 2 (520) at a concentration of 5 μg/μL. UEA1-colloidal gold (UEA1-Au; 300) conjugate was dispensed in the conjugate path (800), which was made of a Whatman R24 membrane. The conjugate path was half covered from the bottom with the sample pad (100), made of a Whatman filter paper no. 3. On the top, a wick path (310) was added to absorb the remaining fluid from the membrane.

Saliva samples (110) from infants were collected and each pre-dissolved in a tube containing a mucolytic solution (N-acetyl cysteine). Each sample was then added onto the sample pad of the device described above and presence/absence of color bands at zone 1 (420) and zone 2 (520) was examined 15 minutes later. If a color band was visible at both zone 1 and zone 2, the saliva sample was determined as secretor glycan positive. If a color band was visible only at zone 2, the saliva sample was determined as secretor glycan negative. See FIG. 2.

The secretor status of the infants participated in this study was determining following the method described above. The data indicates that the infants who are secretor glycan negative are resistant to diarrhea, and those who express high levels of secretor glycan in saliva have a high risk of diarrhea. The data also indicates that secretor-positive infants have 2.5-fold increased risk of moderate-to-severe diarrhea compared to secretor-negative infants (p=0.02). Secretor-positive infants who are breastfed have significantly greater protection against diarrhea if their maternal milk contains high quantities of secretor glycan. Thus, the secretor genotype and salivary phenotype of term infants and their mothers are biomarkers for infant risk of diarrhea.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A device for detecting a glycan, comprising
a sample pad,
a membrane in communication with the sample pad,
a conjugate containing a first lectin and a label,
an immobilized second lectin located at a first zone on the membrane, the second lectin being identical to the first lectin, and
an immobilized antibody specific to the lectin, the antibody being located at a second zone on the membrane,
wherein the first zone and the second zone are separated and the conjugate is located between the sample pad and the first or second zone.

2. The device of claim 1, wherein the membrane is selected from the group consisting of a nitrocellulose membrane, a nylon membrane, a cellulose membrane, a polyvinuylidine fluoride membrane, a polycarbonate membrane, a polypropylene membrane, a polyethylene membrane, a Teflon membrane, and a Kevlar membrane.

3. The device of claim 2, wherein the membrane is a nitrocellulose membrane.

4. The device of claim 1, wherein the lectin is selected from the group consisting of UEA1, AIA, GSA II, WGA, sWGA, SNA, MAL-II, PWA, SJA, LEA, and I-PHA.

5. The device of claim 4, wherein the lectin is UEA1.

6. The device of claim 5, wherein the membrane is a nitrocellulose membrane.

7. The device of claim 1, wherein the label is selected from the group consisting of colloidal gold, fluorescein, rhodamine, green fluorescent protein, quantum dot, and chromophore.

8. The device of claim 7, wherein the label is colloidal gold.

9. The device of claim 1, further comprising a sink pad in communication with the membrane, wherein the sample pad and the sink pad is separated by the membrane.

10. The device of claim 1, further comprising a support member, on which the sample pad and the membrane are mounted.

11. The device of claim 10, further comprising a sink pad, wherein the sample pad, the membrane, and the sink pad are mounted sequentially on the support member.

12. The device of claim 11, wherein the device contains sequentially the sample pad, the conjugate, the immobilized second lectin, the antibody specific to the lectin, and the sink pad.

13. The device of claim 12, wherein the lectin is selected from the group consisting of UEA1, AIA, GSA II, WGA, sWGA, SNA, MAL-II, PWA, SJA, LEA, and I-PHA.

14. The device of claim 13, wherein the lectin is UEA1.

15. The device of claim 10, wherein the support member is plastic or metallic.

16. The device of claim 15, wherein the support member is made of styrene.

17. A method of identifying a glycan expression phenotype in a subject, comprising:
dispensing a bodily fluid from a subject into the sample pad in the device of claim 1,
examining a signal at the first and second zones in the device, and
determining a glycan expression phenotype in the subject based on presence or absence of the signal at the first and second zones,
wherein (i) presence of the signal at both the first zone and the second zone indicates that the subject expresses a glycan specifically binding to the lectin contained in the device of claim 1 and (ii) presence of the signal at only the second zone indicates that the subject does not express the glycan.

18. The method of claim 17, wherein the lectin contained in the device of claim 1 is selected from the group consisting of UEA1, AIA, GSA II, WGA, sWGA, SNA, MAL-II, PWA, SJA, LEA, and I-PHA.

19. The method of claim 18, wherein the lectin is UEA1 and (i) presence of the signal at both the first zone and the second zone indicates that the subject has a secretor glycan-positive phenotype and (ii) presence of the signal only at the second zone indicates that the subject has a secretor glycan-negative phenotype.
